(19) United States
(12) Patent Application Publication
(10) Pub. No.: US 2002/0072127 A1
(43) Pub. Date: Jun. 13, 2002

(54) Assay of chemical binding

(76) Inventors: Carl John Sofield, Rowstock (GB); George Richard Morgan, Harwell (GB); Ruth Elizabeth Harper, Rowstock (GB); Gavin John Stockford, Oxford (GB)

Correspondence Address: LAW OFFICES OF WILLIAM H. HOLT, Unit 2, First Floor, 1423 Powhatan Street, Alexandria, VA, 22314, US

(21) Appl. No.: 09/734,664

(22) Filed: Dec. 13, 2000

(51) Int. Cl. G01N 33/543

(52) U.S. Cl. 436/518

(57) ABSTRACT

A method of comparing the binding strengths of a plurality of different ligands to a receptor, in which several micro-cantilever structures (10) are coated with the receptor on at least a part of a surface (13) of each micro-cantilever structure (10). Each micro-cantilever structure (10) is then contacted with a different ligand solution, and the amounts by which the micro-cantilever structures deflect are compared. The deflection may be detected by an optical lever (16,18; 28). The micro-cantilever structures (10) may be in the form of an array, each structure (10) being in a respective well (24), to which ligand solutions are added.

This invention relates to a method and an apparatus for assaying chemical binding, that is to say reversible reactions between a receptor and a ligand.

Micro-cantilevers such as those used in atomic force microscopy have recently been suggested for use in other applications, for example being sensitive to temperature. It has been suggested that measurements may be based on either the frequency of vibration, or the bending of the micro-cantilever. For example the force of adhesion between the tip of a micro-cantilever derivatized with avidin, and agarose beads functionalized with biotin, has been measured by Florin et al. (Science, April 1994, 264 p. 415). The use of a micro-cantilever to observe changes in surface stress has been described by H. J. Butt (Journal of Colloid and Interface Science 180 (1996) pp. 251-260). A micro-cantilever will bend if the surface stress on one face changes, and this change might for example be caused by a change of pH or of salt concentration if one face of the micro-cantilever is coated with a different material to the opposite face. Butt suggests that such a micro-cantilever may be used to monitor concentrations of substances in the medium around the cantilever, or to measure the specific binding of ligands to cantilevers which are coated on one side with a receptor. However he advises that any such measurements should be performed in a flow-through manner, and his measurements (for example with changing pH) indicate that there is a delay of some minutes before the micro-cantilever responds, so such a process would require significant quantities of reagents; furthermore the relationship with concentration is not clear, as the bending was observed to depend linearly on salt concentration, but also to vary approximately linearly with pH—which is a logarithmic function of concentration.

According to the present invention there is provided a method of comparing the binding strengths of a plurality of different ligands to a receptor, the method comprising coating a plurality of micro-cantilever structures with the receptor, the coating being applied to at least a part of a surface of each micro-cantilever structure, contacting each micro-cantilever structure with a different ligand solution, and comparing the amounts by which the micro-cantilever structures deflect when contacted with the respective ligand solutions.

This method enables you to determine which of the ligands binds most strongly to the given receptor, and so to assay the different ligands in relation to that receptor. Hence the method enables specific binding with any one of the ligands, if it occurs, to be detected, so enabling that ligand to be identified. Equally it enables the binding of the different ligands to the receptor to be ranked in order of strength. Measurements of deflection with different concentrations of the same ligand may also enable the equilibrium constant, K, for the binding reaction to be determined.

The invention also provides an apparatus for performing this method, the apparatus incorporating an array of such micro-cantilever structures, and means to measure how much the structures deflect. Deflection of the structures may be detected optically, for example by reflecting light from a reflective portion of the micro-cantilever structure onto a position-sensitive photodiode.

The micro-cantilever structures are desirably all of the same size. Each may be in the form of a rectangular strip of length less than 0.5 mm, and typically of length between 0.1 and 0.4 mm, fixed at one end, and of thickness typically less than 0.001 mm, for example 0.6 microns. They may be of V shape, and the width of each arm (or of the cantilever) is typically less than a fifth of its length. They may be made of materials such as silicon nitride, silicon, or polymers. Because of their small size their natural frequency of vibration can exceed 10 kHz, so they respond rapidly and are not much affected by noise (which tends to be of lower frequencies). The coating of such micro-cantilevers with a chromium layer followed by a gold layer is known, this improving the optical reflectivity of the coated surface so that deflection or bending of the micro-cantilever can be detected optically, for example with an optical lever. The gold layer can be further coated with organic chemicals, for example with long-chain alkanethiols (e.g. octadecanethiols, as such thiols form self-assembled, highly ordered, stable monolayers on gold.

A potential problem with such micro-cantilevers is that temperature changes can also cause bending. This may be prevented by ensuring the temperature does not change significantly during measurements. Preferably each micro-cantilever structure incorporates means to enable deflections due to ligand-receptor binding to be distinguished from those due to other causes of bending (such as vibration, or temperature). Such common mode noise rejection may be achieved using a V shape micro-cantilever coated with the organic receptor material on just one arm, so that ligand-receptor binding causes twisting of the micro-cantilever rather than bending and temperature changes cause bending rather than twisting). Such twisting has a higher resonant frequency than bending, which further suppresses the effect of noise. Twisting may be detected more easily by providing a cross piece integral with the V shaped micro-cantilever. An alternative embodiment uses two adjacent rectangular micro-cantilevers whose free ends are linked by a torsion bar. Another alternative uses two adjacent micro-cantilevers just one of which is coated with the organic receptor material, and the difference in the bending of the two micro-cantilevers is determined.

The micro-cantilevers might be coated with organic receptor molecules using the thiol approach described previously, or by means of an interposed bonding layer as described in GB 2 225 963 B. The bonding layer may comprise a silylating reagent, such as R₃Si(CH₂)NH₂, which can be connected to a protein by bonding the amino group (—NH₂) to a carboxylic acid group; as in that patent, R can be O-alkyl, O-aryl, O-heterocyclic, alkyl, aryl, or heterocyclic, and n may be zero or any integer. The receptor molecules may then be treated with an initial ligand of moderate binding strength such that only those ligands under test which bind more strongly (and therefore displace the initial ligand) cause deflection of the micro-cantilever structure.

Different coating processes may be suitable for applying other receptor molecules. For example the coating may be deposited by Langmuir-Blodgett film transfer, which forms a monomolecular layer.

The method may comprise arranging an array of micro-cantilever structures so that each micro-cantilever structure is immersed in a respective vessel of water, and then adding solutions of the ligands to each of the vessels and observing the effect on each of the micro-cantilever structures. The addition may cause vibration of the micro-cantilever, but this is only transient. Thus by using an array of for example 200 micro-cantilever structures and 200 corresponding vessels, the strength or bonding of 200 different potential ligands to a given receptor can be compared simultaneously. Each vessel must be large enough to accommodate a micro-cantilever structure, but can therefore be as small as a 1 mm cube, or even smaller. In an alternative method each micro-cantilever structure is in a respective flow channel, through which different solutions are caused to flow.

The deflection of each micro-cantilever structure can be related to the equilibrium constant K for the ligand-receptor binding reaction, and hence to the change of free energy for that reaction. The rate constant for adsorption cannot usually be measured, because the rate of change of reflection of the micro-cantilever structure is usually limited by the diffusion of ligand through the water rather than by the rate of adsorption; however if the concentration is sufficiently high and the size of the vessel is sufficiently small then the rate of adsorption can also be determined. The rate constant for desorption may be measured using the flow channel method, initially contacting a micro-cantilever with a ligand solution so that binding occurs, and then contacting it with pure water so that desorption can occur (the water flushing away any desorbed ligand molecules).

The invention will now be further and more particularly described, by way of example only, and with reference to the accompanying drawings, in which:

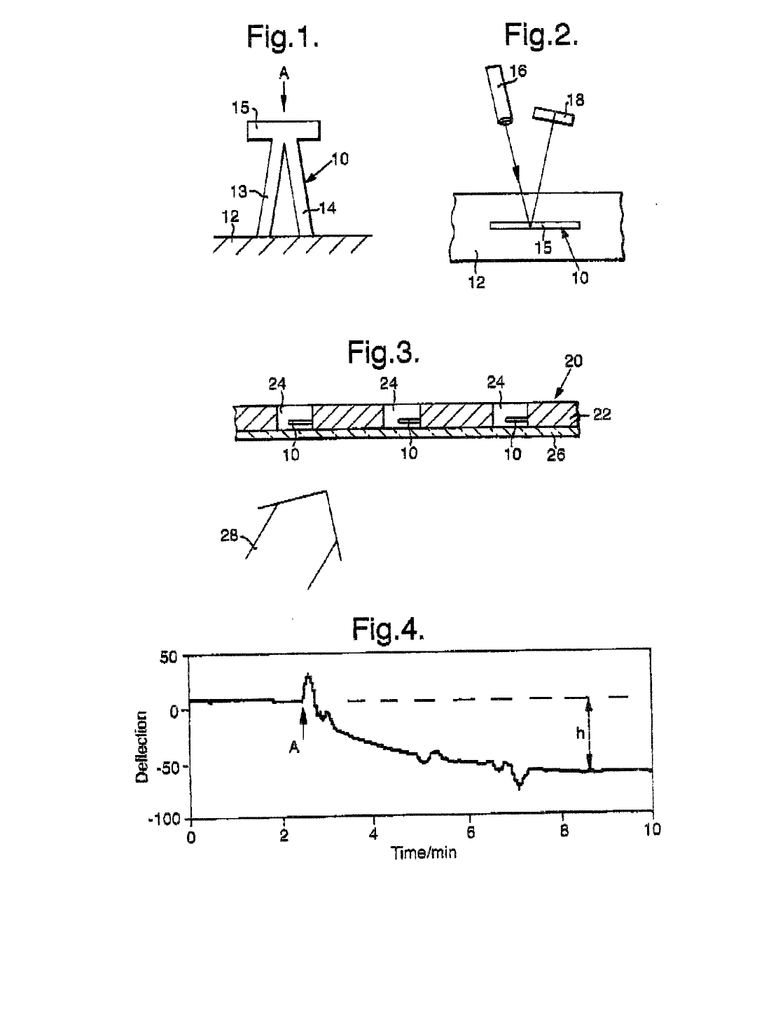

FIG. 1 shows a plan view of a micro-cantilever;

FIG. 2 shows a view in the direction of arrow 2 of FIG. 1;

FIG. 3 shows a sectional view of apparatus incorporating an array of micro-cantilevers;

FIG. 4 shows graphically the variation of deflection with time of a micro-cantilever as a result of a ligand-receptor binding reaction;

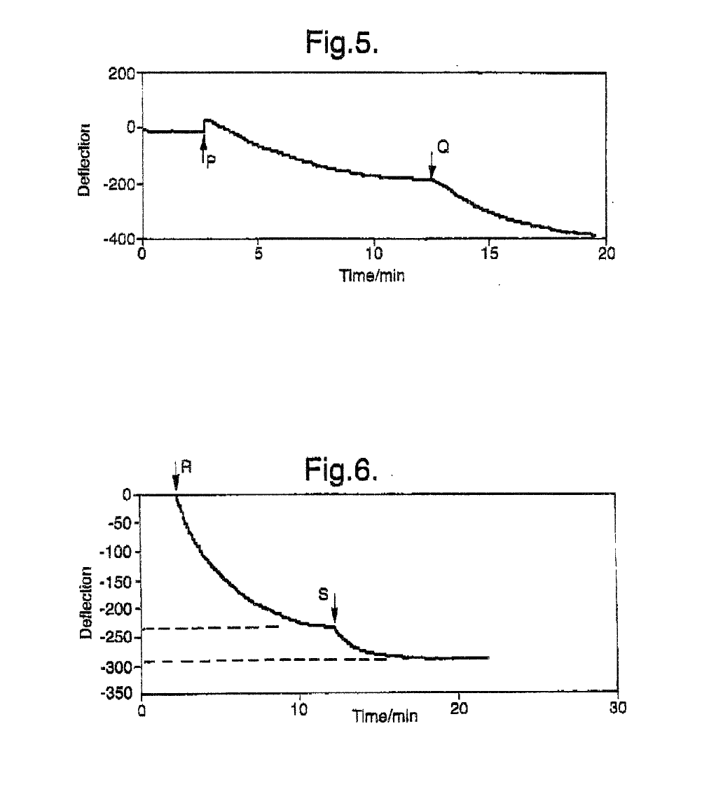

FIG. 5 shows graphically the variation of deflection with time where two different ligands bind successively to a receptor; and

FIG. 6 shows graphically the variation of deflection with time where a micro-cantilever is exposed to different concentrations of a ligand.

Referring to FIG. 1, a micro-cantilever 10 is fixed at one end to a block 12. The micro-cantilever 10 is generally V-shaped in plan, comprising two converging strips 13,14, which are integral with a transverse cross strip 15. It projects 0.2 mm from the block 12, the strips 13 and 14 each being 24 microns wide and the entire micro-cantilever 10 is of silicon nitride of thickness 0.6 microns. The top surface of the micro-cantilever 10 is coated with a 5 nm layer of chromium followed by a 13 nm layer of gold, to improve its optical reflectivity. The gold on one strip 13 is then coated with octadecanethiol, which forms a self-assembled, highly ordered monolayer on gold, and biotin is then bonded to this monolayer. Biotin acts as a selective receptor for avidin.

Application of a coating of receptor molecules on just one strip 13 may be achieved by coating just that strip with gold (by masking the other strip 14); or by coating both strips 13 and 14 with gold and with receptor molecules, andrd then removing the receptor molecules from one strip 14 for example by ozone and ultraviolet irradiation, or by using a laser (with masking of the other strip 13).

Referring now to FIG. 2 any deflection or twisting of the micro-cantilever 10 is detected optically, by focusing a light beam from a laser diode 16 onto the cross strip 15, and detecting the reflection with a quadrant photodiode 18. The light intensities detected by the four segments of the photodiodo 19 may be used to determine the deflection of the cross strip 15. If the micro-cantilever 10 is exposed to a solution of avidin, which binds to the biotin, this changes the surface stress of the strip 13, causing the micro-cantilever 10 to twist, so changing the inclination of the cross strip 15.

Referring now to FIG. 3, test equipment 20 comprises a silicon wafer 22 defining an array of through holes 24 each of diameter 0.7 mm. Within each hole 24 is a micro-cantilever 10 with gold on its lower surface. A thin glass plate 26 is bonded to the lower surface of the wafer 22, so that an array of 1iquid vessels are defined by the holes 24 and the plate 26. Deflection of the micro-cantilevers 10 is detected by shining a beam of light onto the lower surface of the glass plate 26, and detecting the movement of the reflected spots of light. In use of the equipment 20, the gold surface of each micro-cantilever 10 is coated with the same receptor (e.g. biotin), and water is placed in each vessel 24. Solutions of different ligands are then injected into each vessel, a different ligand into each vessel, so that the degree to which each ligand bonds to that receptor can be ranked.

Referring now to FIG. 4, this shows graphically the variation with time of the deflection (in arbitrary units) of the micro-cantilever 10 when immersed in water in a small vessel of volume 0.4 ml, before and after injecting avidin into the water. After injection, the concentration of avidin is 0.08 μM. The time at which the avidin is injected is indicated by the arrow A. Before this time the deflection is substantially constant. The gradual change in deflection over a period of about five minutes, the deflection then reaching a new, steady value differing by h from its initial value. The bending is caused by the surface stress change resulting from the reaction; the change in surface stress is, at least approximately, equal to the change of surface energy, which can be related to the concentration c or the ligand and the equilibrium constant K of the binding reaction onto the surface (i.e. ka/kd, where ka is the adsorption coefficient and kd is the desorption coefficient). Consequently the deflection h is given by:

h=C 1n( 1+ck)

where C is a constant.

Referring now to FIG. 5, this shows graphically the variation with time of the deflection (in arbitrary units) of the micro-cantilever 10 when immersed in water in the small vessel. At the time indicated by the arrow P immunoglobulin G (IgG) was injected, and at the time indicated by the arrow Q avidin was injected. As with the results shown in FIG. 4, after each injection the deflection gradually changes over a period of several minutes before reaching a new steady value. In this case the deflection resulting from the immunoglobulin was about 200 units, and addition of avidin—which binds to biotin more strongly—led to a further deflection of about 200 units. The avidin displaces the immunoglobulin G which has bound to the biotin, because it binds more strongly.

Referring now to FIG. 6, this shows graphically the variation with time of the deflection (in arbitrary units) of the micro-cantilever 10 when immersed in water in the small vessel. At the time indicated by the arrow R immunoglobulin G (IgG) was injected so the concentration in the vessel was 0.047 μM, and at the time indicated by the arrow S additional immunoglobulin G (IgG) was injected to raise the concentration to 0.068 μM. The deflection resulting from the initial injection (after about 10 minutes) was about 235 units, whereas the deflection resulting from the second injection was about 290 units (after a further 10 minutes). As expected from the equation for h given above, in a case such as this where the equilibrium constant is large (say 10⁹ l/mole), the deflection does not increase linearly with concentration.

It will be appreciated that the process might be modified in various ways while remaining within the scope of the invention. A variety of different ways of bonding the receptor to the micro-cantilever structure may be used instead of the long chain alkane thiol approach described previously. An alternative bonding molecule would comprise a long chain alkane having a thiol group near one end and a carboxylic acid group near the other end, i.e. COOH-R-SH, where the thiol group would be bonded to the gold layer; the carboxylic acid group might then be bonded to a protein. An alternative bonding layer is a silylating reagent as described in GB 2 225 963 B. The micro-cantilever structures might be of a different shape to that described above, for example comprising two adjacent rectangular micro-cantilevers whose free ends are linked by a torsion bar; movement of the torsion bar might be detected optically or capacitively.

It is desirable to coat the surface of the micro-cantilever 10 opposite that on which is the coating of receptor molecules, to suppress any potential biochemical interactions at that surface. Diamond-like carbon is a suitable coating for this purpose. This can for example be deposited, in a vacuum chamber, by exposing those surfaces to a vapour of a hydrogenated carbonaceous material (such as polyphenyl ether) while subjecting the surfaces to bombardment by ions of say oxygen or nitrogen of energy in the range 40-80 keV.

We claim:

1. A method of comparing the binding strengths of a plurality of different ligands to a receptor, the method comprising coating a plurality of micro-cantilever structures with the receptor, the coating being applied to at least a part of a surface of each micro-cantilever structure, contacting each micro-cantilever structure with a different ligand solution, and comparing the amounts by which the micro-cantilever structures deflect when contacted with the respective ligand solutions, in the absence of any vibration of the micro-cantilever structures.

2. A method as claimed in claim 1 wherein each micro-cantilever structure is of length less than 0.5 mm, of thickness less than 0.001 mm, and is fixed at one end.

3. A method as claimed in claim 1 wherein each micro-cantilever structure is of V shape, and the width of each arm is less than a fifth of its length.

4. A method as claimed in claim 3 wherein the organic receptor is coated on just one arm of each micro-cantilever structure, so that ligand-receptor binding causes twisting of the micro-cantilever structure.

5. A method as claimed in claim 1 wherein each micro-cantilever structure comprises two adjacent rectangular micro-cantilevers whose free ends are linked by a torsion bar.

6. A method as claimed in claim 1 wherein each micro-cantilever structure is coated with the receptor by means of an interposed bonding layer.

7. A method as claimed in claim 6 wherein the bonding layer comprises R₃Si(CH₂)ₙN₂, wherein R is O-alkyl, O-aryl, O-heterocyclic, alkyl, aryl, or heterocyclic, and n is zero or any integer.

8. A method as claimed in claim 1 wherein the receptor is treated with an initial ligand of moderate binding strength, such that only those ligands under test which bind more strongly cause deflection of the micro-cantilever structure.

9. A method as claimed in claim 1 comprising arranging an array of micro-cantilever structures so that each micro-cantilever structure is immersed in a respective vessel of water, and then adding solutions of the ligands to each of the vessels and observing the deflection of each of the micro-cantilever structures.

10. A method as claimed in claim 9 wherein the deflection is measured optically.

11. An apparatus for comparing the binding strengths of a plurality of different ligands to a receptor, the apparatus comprising a plurality of micro-cantilever structures coated with the receptor, the coating being applied to at least a part of a surface of each micro-cantilever structure, means for contacting each micro-cantilever structure with a different ligand solution, and means for comparing the amounts by which the micro-cantilever structures deflect when contacted with the respective ligand solutions in the absence of any vibration of the micro-cantilever structures.

12. An apparatus as claimed in claim 11 wherein each micro-cantilever structure is of length less than 0.5 mm, of thickness less than 0.001 mm, and is fixed at one end.

13. An apparatus as claimed in claim 11 wherein each micro-cantilever structure is of V shape, and the width of each arm is less than a fifth of its length.

14. An apparatus as claimed in claim 13 wherein the organic receptor is coated on just one arm of each micro-cantilever structure, so that ligand-receptor binding causes twisting of the micro-cantilever structure.

15. An apparatus as claimed in claim 11 wherein each micro-cantilever structure comprises two adjacent rectangular micro-cantilevers whose free ends are linked by a torsion bar.

16. An apparatus as claimed in claim 11 wherein each micro-cantilever structure incorporates an interposed bonding layer onto which the receptor is coated.

17. An apparatus as claimed in claim 16 wherein the bonding layer comprises R₃Si(CH₂)ₙNH₂, wherein R is O-alkyl, O-aryl, O-heterocyclic, alkyl, aryl, or heterocyclic, and n is zero or any integer.

18. An apparatus as claimed in claim 11 comprising at least one light source arranged to generate a beam of light incident on at least one of the micro-cantilever structures, and at least one sensor to detect movement of the light beam reflected from the said micro-cantilever structure.
